(19) United States
(12) Patent Application Publication
(10) Pub. No.: US 2023/0285685 A1
(43) Pub. Date: Sep. 14, 2023

(54) Safety Device for a Needle

(71) Applicant: Becton Dickinson France, Le Pont de Claix (FR)

(72) Inventors: Olivier Alvain, Seyssins (FR); Frédéric Michel, Rives (FR); Franck Carrel, Saint Jean de Vaulx (FR); Julien Singer, Domene (FR)

(21) Appl. No.: 18/011,267

(22) PCT Filed: Jun. 22, 2021
(86) PCT No.: PCT/EP2021/067041

§ 371 Date: Dec. 19, 2022

(30) Foreign Application Priority Data
Jun. 23, 2020 (EP) 20315314.3

(51) Int. Cl. A61M 5/32; A61M 5/50

(52) U.S. Cl. CPC A61M 5/3243; A61M 5/3278; A61M 5/50

(57) ABSTRACT

A safety device for a needle of a medical device, the safety device including a ring adapted to be fixed with regard to the needle, a shield rotatably fixed on the ring, and the shield being configured to at least rotate in a closing direction from a retracted position in which the shield does not cover the needle to a safety position in which the shield covers the needle. The shield includes a proximal portion and a distal portion linked to the proximal portion so as to be rotatable from an operating position to a tilted position in which the distal portion is tilted with regard to the proximal portion.

### CROSS REFERENCE TO RELATED APPLICATIONS

This application is the United States national phase of International Patent Application No. PCT/EP2021/067041 filed Jun. 22, 2021, and claims priority to European Patent Application No. 20315314.3 filed Jun. 23, 2020, the disclosures of which are hereby incorporated by reference in their entireties.

### BACKGROUND OF THE INVENTION

### Field of the Invention

The present invention relates to a safety device adapted for a needle, a safety needle hub and a medical device such as a syringe comprising such a safety device.

### Description of Related Art

A wide number of medical devices rely on a needle in order to prick a patient’s skin and to deliver a medicine or to collect a body fluid. In particular, syringes are a common way of delivering drugs or vaccines to patients and usually comprise an injection needle to deliver such drugs into a patient’s skin or a patient’s muscle. However, needles present a sharp tip with a risk of needle stick injury, in particular for the medical staff, and it is highly desirable to prevent such needle stick injury for safety reasons.

Consequently, safety devices have been proposed in order to cover the needle when the needle is not to be used and to allow access to the needle when a medical act is performed. For example, document EP3302656 discloses such a safety device with a pivoting shield that pivot to a safety position covering the needle, thanks to a simple push on the shield, thus allowing a safe handling and disposal of the syringe after use.

However, it appears that medical staff may be confused by the different kinds of safety devices existing on the market, some of them requiring a different handling to reach their safety position. In particular, some of them require to press the shield against a flat surface in order to bend the needle. However, if the shield of a safety device requiring a simple push to reach the safety position is actually pressed against a flat surface, the needle may be bent to an inappropriate angle and may then escape from the shield in the safety position, thus rendering the safety device inefficient because of such a misuse.

There is thus a need to propose a safety device for a needle of a medical device which allows the needle to be efficiently covered by a shield in the safety position, even if the appropriate operating instructions of the safety device are not followed.

### SUMMARY OF THE INVENTION

This objective is accomplished by a safety device for a needle of a medical device, the safety device comprising:
a ring adapted to be fixed with regard to the needle,
a shield rotatably fixed to the ring, the shield being configured to at least rotate in a closing direction from a retracted position in which the shield does not cover the needle to a safety position in which the shield covers the needle,
wherein the shield comprises a proximal portion and a distal portion linked to the proximal portion so as to be rotatable from an operating position to a tilted position in which the distal portion is tilted with regard to the proximal portion.

Thanks to the rotatable distal portion, the shield can cover the needle even if the present safety device is not used appropriately. Consequently, the present safety device offers a higher level of safety for users and patients and contributes to reduce or prevent needle stick injuries.

Advantageously, the safety position includes a first safety position in which the shield covers the needle and is in the operating position and a second safety position in which the shield covers the needle and is in the tilted position. The user has thus the choice between two different safety positions which prevents any misuse of the present safety device.

Advantageously, the proximal portion and the distal portion of the shield define a recess adapted to accommodate the needle in the safety position. Thanks to the recess extending in both the proximal portion and the distal portion of the shield, the needle may be efficiently covered in any safety positions.

Advantageously, the proximal portion and the distal portion are linked by a pivot link adapted to face or contact the needle at least in the first safety position of the shield.

Advantageously, the distal portion is configured to rotate with regard to the proximal portion when a force equal or greater than an activation force is applied on said distal portion. This allows the present safety device to appear and behave similarly to a safety device according to the prior art while allowing a rotation of the distal portion toward the tilted position in case of a misuse.

Advantageously, the distal portion is configured to rotate with regard to the proximal portion in the closing direction, which corresponds to a usual misuse and may render the present safety device more user friendly.

Advantageously, the distal portion is linked to the proximal portion by a living hinge i.e. a refined or thinned portion of the shield formed as an integral part, which is a cost-effective and easy to manufacture type of hinge. Preferably, the living hinge may be configured to be permanently deformed by the rotation of the distal portion, which can increase the safety level of the present safety device by limiting the risk of not covering the needle tip in the safety position, even in the case of a misuse.

Advantageously, the shield and the ring comprise locking members configured to lock the shield to the ring in the safety position. Such a locked shield can cover permanently the needle after use and offers a high level of safety.

Advantageously, the locking members comprise at least one dead stop defined on the ring and at least one moving stop defined on the proximal portion of the shield and the moving stop is configured to be on a first side of the dead stop in the retracted position of the shield and to move to a second side of the dead stop when the shield is rotated to the safety position. Such locking members offer a high level of safety and are easy to manufacture, for example as appropriate shapes of the ring and the shield. For example, the moving stop is configured to be flexed or deflected by the dead stop when it moves from the first side to the second side of the dead stop.

Preferably, the dead stop can be located on a portion of the ring facing a direction opposite the closing direction of the shield i.e. an opening direction, which allows for a safe and user-friendly movement of the shield from the retracted position to the safety position. In other words, the dead stop can be located or defined on a portion of the ring which is covered by the shield in the retracted position of the shield, for example by lateral surfaces of the shield.

Advantageously, the shield has a longitudinal axis and the distal portion has a length along the longitudinal axis of half or less than half a length of the proximal portion along the longitudinal axis and for example at least one sixth or one fifth of the length of the proximal portion along the longitudinal axis. Such a configuration may allow the shield to efficiently covers the needle even after a misuse. For example, the length of the distal portion may be one third of the length of the proximal portion.

Advantageously, the ring is centred on a central axis and the shield is configured so that a longitudinal axis of the distal portion forms an angle of 75 to 105°, preferably of 80 to 100° and again preferably 90° with the central axis of the ring, when the shield is in the safety position and the distal portion is in the tilted position. Such a configuration may allow to offer a high level of safety for the user, even during and after a misuse of the present safety device, such as a pressure of the distal portion on a surface.

Preferably, the longitudinal axis of the proximal portion may form an angle with the central axis of the ring of 10 to 45°, preferably 15 to 40° and again preferably 25°, when the shield is in the safety position, which allows for an easy handling of the safety device.

Advantageously, the shield comprises a thinned portion provided between the proximal portion and the distal portion which can provide a cost-efficient contribution to the rotatable or pivot link of the distal portion to the proximal portion. For example, this thinned portion may be comprised in the living hinge or may define the living hinge.

Advantageously, the shield comprises a main surface and at least a lateral surface, the thinned portion being provided at least on the lateral surface and being configured to fold when the distal portion rotates. Such a thinned surface may thus allow to mask the pivot link between the distal portion and the proximal portion to the view of the user and thus may avoid the user to believe that the tilted position of the distal portion is actually recommended. Preferably, two lateral surfaces are provided, each of them defining a thinned portion and the shield can have a U-shaped cross section.

Preferably, the main surface defines a flat outside surface facing the opening direction, at least on the distal portion of the shield, which may allow the user to safely rotate the shield from the retracted position to the safety position and may offer a high level of safety to the user during a misuse.

A second aspect of the invention is a safety needle hub adapted to be fixed on the tip of a medical device, for example a syringe, the safety needle hub comprising a needle and a safety device according to the first aspect of the present invention.

A third aspect of the present invention is a medical device comprising a needle and a safety device according to the first aspect of the present invention or comprising a safety needle hub according to the second aspect of the present invention.

Advantageously, the needle according to any aspect of the present invention has a tip and the distal portion of the shield is adapted to cover at least the tip of the needle when the shield is in the safety position.

Advantageously, the distal portion according to any aspect of the invention comprises an inside pad adapted to contact the tip of the needle when the distal portion is in the tilted position. For example, the tip of the needle may be inserted in the inside pad in the tilted position and/or in the safety position, thus offering a higher degree of safety for the user or the patient, in particular in case of a misuse.

Preferably, the needle is aligned on the central axis of the ring, which may offer a simple and efficient configuration of the medical device or the safety needle hub.

Advantageously, the distal portion of the shield covers at most a distal half portion, a distal quarter portion or a distal fifth portion of the needle when the shield is in the safety position, which may allow to limit the bending of the needle in the case of a misuse so that the shield may efficiently cover the needle in the safety position. Preferably, at least the needle tip is covered by the distal portion in the safety position of the shield.

A fourth aspect of the present invention is a safety device for a needle of a medical device, the safety device comprising:
a ring adapted to be fixed with regard to the needle,
a shield rotatably fixed to the ring, the shield being configured to at least rotate in a closing direction from a retracted position in which the shield does not cover the needle to a safety position in which the shield covers the needle,
wherein the shield comprises a proximal portion and a distal portion linked to the proximal portion so as to be rotatable, preferably when a force equal or greater than an activation force is applied on said distal portion.

### BRIEF DESCRIPTION OF THE DRAWINGS

Further advantages and preferred embodiments of the present invention will become apparent from the following detailed description and drawings, in which:

FIG. 1 is a side view of a safety device according to the present invention in an initial position and mounted on a syringe as a medical device.

FIG. 2 is a detailed cross section view of the safety device according to FIG. 1.

FIG. 3 is a side view of the safety device of FIG. 1 in a retracted position.

FIG. 4 is a side view of the safety device of FIG. 1 in a safety position.

FIG. 5 is a detailed cross section view of the safety device according to FIG. 4.

FIG. 6 is a detailed side view of the safety device according to FIG. 5 in a tilted position.

FIG. 7 is a detailed cross section view of the safety device of FIG. 6.

### DESCRIPTION OF THE INVENTION

The present safety device is intended to be used with or on any kind of injection, test or sampling medical device using a needle adapted to prick a patient’s body and any kind of prophylactic, diagnosis, aesthetics or therapeutic medical treatment. For example, such a medical device can be a medical syringe or a blood collection tube. The safety device can be delivered mounted on the medical device or as a safety needle hub adapted to be fixed on a neck of the medical device. In addition, the safety device can also be proposed alone, for example for a subsequent mounting on a syringe or on another medical device, depending on the targeted customer.

The safety device according to the present invention is described in the examples of the appended figures as mounted on a medical syringe. As such, in this application, the distal direction must be understood as the direction of injection with reference to the medical injection system, and the proximal direction is the opposite direction, i.e. the direction toward the hand of the user such as a medical caregiver or a patient.

### Description of the Main Embodiment

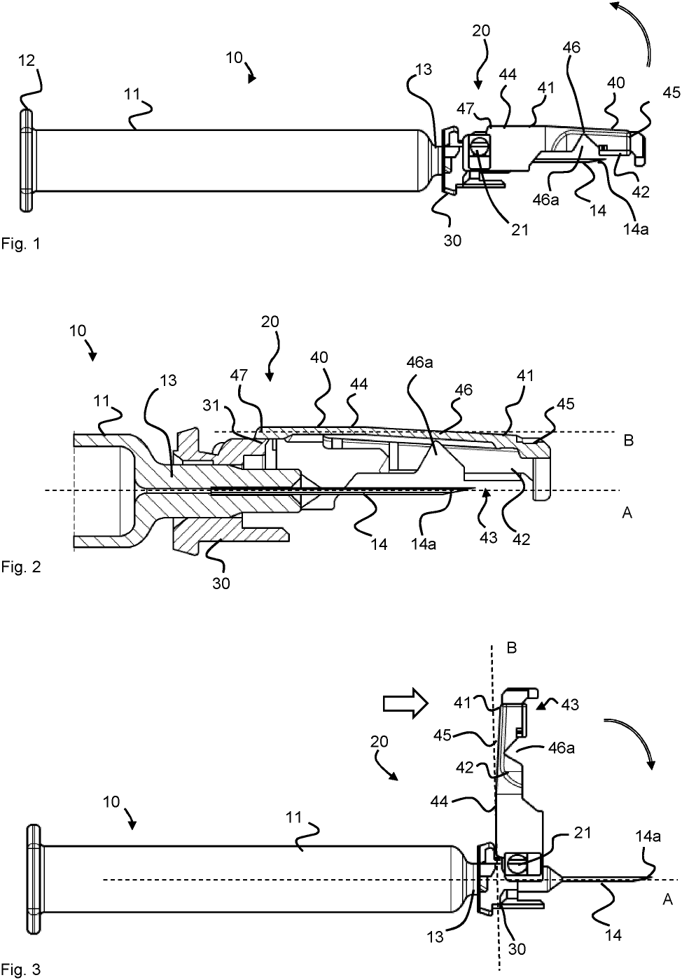

Now referring to FIGS. 1 and 2, and from proximal to distal or from the left side to the right side of FIGS. 1 and 2, a syringe 10 comprises a proximal flange 12, a syringe barrel 11 and a distal neck 13 provided with a needle 14 defining a tip 14a. The needle 14 may be stacked and/or glued to the distal neck 13.

A safety device 20 according to the present invention comprises a ring 30 fixed on the distal neck 13 of the syringe, for example thanks to clipping, gluing, welding and/or friction. The ring 30 can comprise part of a hinge 21 and a dead stop 31 such as a lug pointing in the distal direction and for example provided with a sloped distal surface (see FIG. 2). The ring 30 can be centred on an axis A, such as a revolution axis of the ring 30, and this axis A can be a longitudinal and/or revolution axis of the needle 14 and/or of the syringe 10.

The safety device 20 further comprises a shield 40 rotatably fixed to the ring 30 thanks to a pivot link under the form of a hinge 21 or alternatively a living hinge (not represented). This hinge 21 allows the shield 40 to adopt several positions with regard to the ring 30 and the syringe 10, as described below. In particular, the hinge 21 defines an opening direction of the shield 40 (see the arrow of FIG. 1) and a closing direction of the shield 40 opposite said opening direction. The ring 30 can define a portion facing the opening direction and/or facing the shield 40 when the shield 40 is in the retracted position. The dead stop 31 can be provided on said portion of the ring 30.

With reference to FIGS. 1 and 2, the shield comprises a main surface 41 and two lateral surfaces 42, for example transversal to the main surface 41. The main surface 41 is preferably flat on the outside face facing the opening direction and defines with the lateral surfaces 42 a recess 43 intended to accommodate the needle in the safety position of the shield. The shield 40 can thus have a U-shaped cross section. Further, the shield 40 comprises a proximal portion 44 close to or facing the ring 30 and comprising part of the hinge 21 on the lateral surfaces 42. A distal portion 45 opposite the proximal portion 44 and opposite the ring 30 is also defined on the shield 40. The proximal portion 44 and the distal portion 45 are linked or connected by a pivot link 46, comprising a notch 46a in each of the lateral surfaces 42 of the shield 40.

The pivot link 46 is preferably a living hinge, as shown in FIG. 2, but may also be a hinge, in another embodiment (not represented). The pivot link 46 allows a rotation of the distal portion 45 with regard to the proximal portion 44 to a tilted position, for example when a force or a torque is applied to the distal portion 45, a force equal or greater to an activation force. During a normal operation of the shield 40, the pivot link 46 is not rotated and the distal portion is in an operating position, for example aligned with the proximal portion 44 on a longitudinal axis B of the shield 40. The pivot link 46 may rotate in the closing direction and may resist to a rotation in the opening direction.

The pivot link 46 may be placed on a portion of the shield 40 covering or facing the needle in the initial position and/or in the safety position, such as the first safety position. For example, the pivot link 46 may be comprised in a middle portion of the shield 40 and/or the recess 43 or in a portion between the middle portion and the distal extremity of the shield 40. The shield (40) may have a longitudinal axis (B) and the distal portion (45) may have a length along the longitudinal axis (B) of half or less than half a length of the proximal portion (44) along the longitudinal axis (B).

The shield also comprises a moving stop 47, which can be located on a proximal extremity of the main surface 41 of the shield 40, i.e. on the proximal extremity of the proximal portion 44. This moving stop 47 may be elastically deformable at least toward the opening direction, for example thanks to a reduced thickness and/or thanks to the material of the shield 40. Alternatively, the moving stop 47 may not be integral with the shield but may be a separate part permanently fixed to the shield 40. The moving stop 47 is configured to lie on the dead stop 31 in the initial position and to engage or abut the dead stop 31 in the safety position of the shield 40. For example, the moving stop 47 and/or the dead stop 31 may have sloped surfaces, preferably having a similar angle as visible in the figures.

Finally, the ring 30 and the shield 40 are preferably made of any rigid polymer adapted to medical use, such as high density polyethylene (PE), polypropylene (PP), polycarbonate (PC), acrylonitrile butadiene styrene (ABS), polyoxymethylene (POM), polystyrene (PS), polybutylene terephthalate (PBT), polyamide (PA), and their combinations.

### Operation of the Main Embodiment

In operation, the syringe 10 and the safety device 20 may be provided as visible in FIGS. 1 and 2, wherein the shield 40 is an initial position, partially covering the needle 14. In addition, the distal portion 45 can be in the operating position, not rotated with regard to the proximal portion 44 and for example aligned with the proximal portion 44 along the longitudinal axis B. Thanks to this operating position, the user may not notice any change with regard to a prior art safety device comprising a shield without a pivot link and could thus use the syringe 10 and the safety device 20 according to his/her usual practice.

In addition, a needle cap may surround and protect the needle 14 (not represented in the figures). This needle cap can be removed from the needle 14 before using the syringe 10, as known by the skilled person. In the initial position, the shield 40 can be substantially parallel to the syringe 10. For example, the longitudinal axis B of the shield 40 can be parallel to the central axis A of the ring 30 and/or the syringe 10 (see FIG. 2).

In the initial position of FIGS. 1 and 2, the moving stop 47 is on a first side of the dead stop 31, for example between the central axis A and the longitudinal axis B and is not engaged or locked by the dead stop 31. The shield 40 can thus be rotated with regard to the ring 30 in the opening direction in order to move from the initial position of FIGS. 1 and 2 (see the arrow in FIG. 1) to the retracted position of FIG. 3. The shield 40 may form preferably an angle of at least 45°, preferably 75° and again preferably at least 90° with the syringe in the retracted position, for example between the longitudinal axis B of the shield 40 and the central axis A. In this retracted position, the needle 14 is fully accessible and the syringe 10 can be used, for example to deliver a medicine to a patient according to a usual medical practice.

At the end of the injection operation, the shield 40 can be rotated in a closing direction (see the black arrow of FIG. 3), for example thanks to a force applied by the thumb of the user on the main surface 41 of the shield 40 (see the white arrow in FIG. 3). The dead stop 31 is placed in the path of the moving stop 47 in the closing direction and, according to an operating instruction, an additional force must be applied on the shield 40 so as to deflect the moving stop 47. Such a force may preferably be limited, for example from 1 to 5 N, for example 3 N.

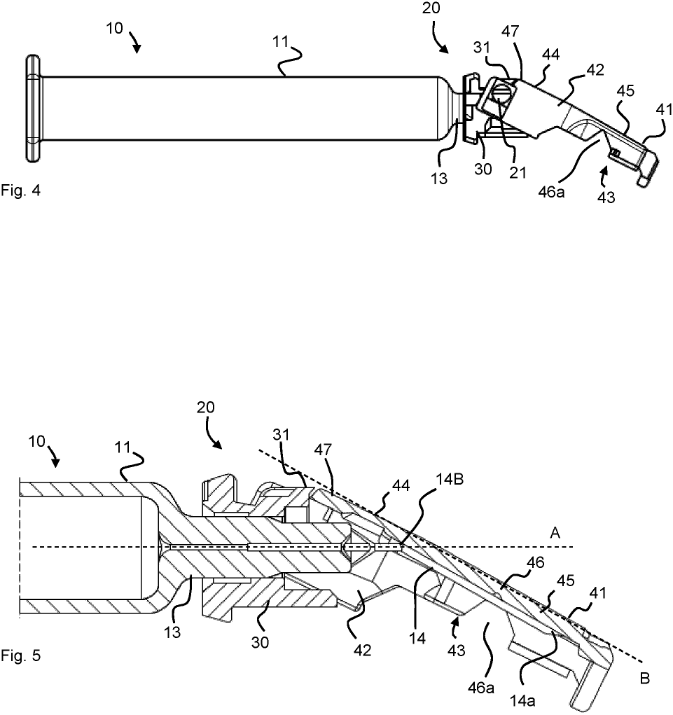

The moving stop 47 can thus reach the second side of the dead stop 31 in an end portion of the closing movement of the shield 40. Consequently, the shield 40 has reached a safety position shown in FIGS. 4 and 5, such as a first safety position and for example form an angle between its longitudinal axis B and the central axis A of the syringe of around 25 ° (see FIG. 5).

With reference to FIG. 5, the moving stop 47 is in abutment with the dead stop 31 of the ring 30, on the other side of the dead stop 31, which prevent any movement of the shield and lock the shield 40 to the ring 30. Consequently, the dead stop 31 and the moving stop 47 act as locking members to lock the shield 40 in the safety position. In addition, the curved or sloped surfaces of the dead stop 31 and the moving stop 47 may be similar or complementary in order to contribute to the locking engagement.

Further, because of the angle made between the shield 40 and the syringe 10, the needle 14 is bent by the rotation movement of the shield 40 for example on a deflection point 14B, in order to render the syringe 10 visibly improper for use. Preferably, a part of the needle 14 can be accommodated in an internal slot of the recess 43 of the shield 40.

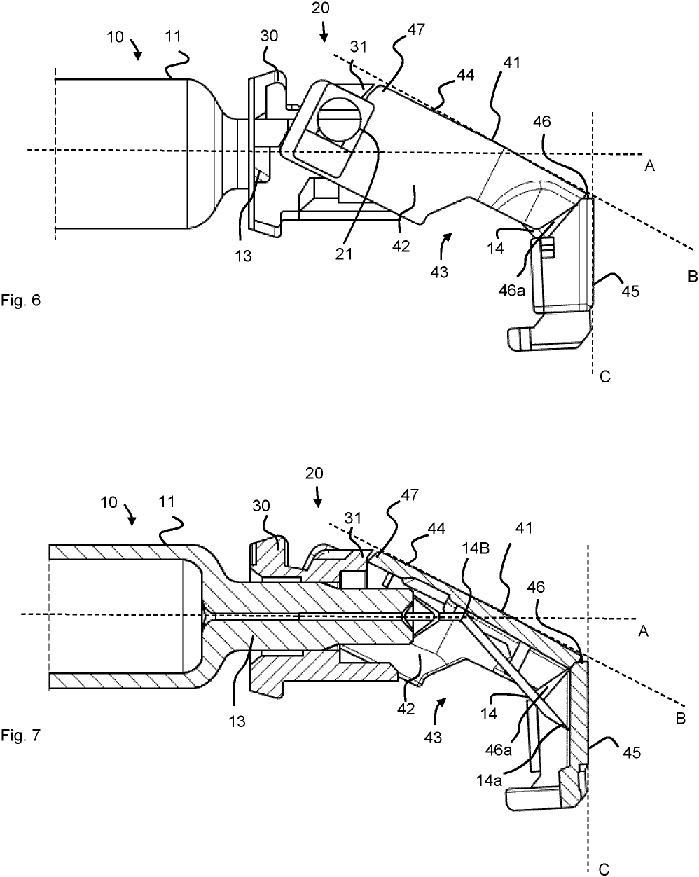

Now with reference to FIGS. 6 and 7, in case of a misuse of the safety device 20, the main surface 41 of the distal portion 45 may be pressed on a flat surface, for example on a table, despite the operating instruction to keep the safety device in the safety position of FIGS. 4 and 5. In this case, the distal portion 45 may rotate with regard to the proximal portion 44 in a tilted position, for example in order to form a 90° or at least a 80° angle between a longitudinal axis C of the distal portion 45 and the central axis A and the shield 40 may be in a second safety position. The force to rotate the distal portion is preferably higher than the force to move the shield to the safety position, for example of 5 to 20 N, preferably 12 N.

Thanks to the pivot link 46, located on or facing the needle in the safety position, an appropriate angle can be maintained between the syringe (axis A) and the proximal portion 44 of the shield 40 (axis B). Consequently, the additional bending of the needle 14 resulting from the misuse (i.e. the distal portion 45 pressed on a surface) can be limited and the deflection point 14B of the needle may not be changed, with regard to the safety position of FIGS. 4 and 5.

Preferably, the pivot link 46 is located at or distally from a middle portion of the shield 40, for example at in a distal third section of the length of the shield 40, which allows to limit the angle made by the bended needle 14 and to prevent the bended needle 14 from escaping from the recess 43. In other words, the distal portion 45 of the shield 40, having a length of half or less than half a length of the proximal portion (44) along the longitudinal axis (B) allows to efficiently cover the needle 14 in the second safety position defined by the tilted shield.

Preferably, the pivot link 46 may allow for a permanent deformation of the shield 40 and the distal portion 45 may be permanently in the tilted position of FIGS. 6 and 7. Such a permanent deformation may be a plastic deformation, for example obtained by a choice of a material having a low elastic limit, such as below the force required to move the distal portion i.e. below 20 N or preferably below 5 N. This embodiment offers a high level of safety as the needle 14 is not accessible to an user after use: needle stick injuries are limited or prevented as well as any undesired reuse of the syringe 10.

### Other Embodiments

In another embodiment, the pivot link 46 of the shield 40 can have an elastic behaviour and the distal portion 45 can return at least partially to its operating position (i.e. aligned with the proximal portion or close from this alignment). However, the deflection of the needle 14 has been limited by the rotation of the distal portion 45 and the tip 14a of the needle 14 may be kept within the recess 43 or at least covered by the shield 40, even if the distal portion 45 returns in the operating position. Consequently, such a safety device can maintain an appropriate level of user protection despite the misuse.

Further, an internal pad (not represented) may be provided inside the recess 43 in order to contact or to be pricked by the tip of the needle in the tilted position of the distal portion 45 and/or in the safety position of the shield 40, in order to further increase the safety level of the safety device according to the present invention.

In another embodiment, the notch 46a may be filled with thin material and for example consists in a thinned portion of the shield or be masked with a label or a thin film of polymer. For example, the thinned portion has a thickness of 0.10 to 0.20 mm, preferably 0.15 mm and can consist in polyoxymethylene (POM). Alternatively or in combination, the lateral surface of the distal portion can be shifted with regard to the lateral surface of the proximal portion and the notch 46a can then be a slot. These embodiments are valuable to avoid the user thinking that the tilted position of the distal portion is appropriate according to the operating instructions.

The locking members may also have a different design, for example with stepped surfaces and/or be located in another portion of the ring and of the shield, for example on the lateral sides of the ring and the shield, i.e. around the hinge 21.

All these embodiments can be combined together and/or with the main embodiment.

Although the present invention has been described and illustrated in detail, it is clearly understood that the same is by way of illustration and example only and is not to be taken by way of limitations, the scope of the present invention being limited only by the terms of the appended claims.

1. A safety device for a needle of a medical device, the safety device comprising:
a ring adapted to be fixed with regard to the needle, and
a shield rotatably fixed to the ring the shield being configured to at least rotate in a closing direction from a retracted position in which the shield does not cover the needle to a safety position in which the shield covers the needle,
wherein the shield comprises a proximal portion and a distal portion linked to the proximal portion so as to be rotatable from an operating position to a tilted position in which the distal portion is tilted with regard to the proximal portion,
wherein the distal direction is the direction of injection and the proximal direction is the opposite direction, and
wherein the safety position includes a first safety position in which the shield is in the operating position and a second safety position in which the shield is in the tilted position.

2. The safety device according to claim 1, wherein the proximal portion and the distal portion of the shield define a recess adapted to accommodate the needle in the safety positions.

3. The safety device according to claim 1, wherein the proximal portion and the distal portion are linked by a pivot link adapted to face or contact the needle at least in the first safety position of the shield.

4. The safety device according to claim 1, wherein the distal portion is configured to rotate with regard to the proximal portion when a force equal or greater than an activation force is applied on said distal portion.

5. The safety device according to claim 1,wherein the distal portion is configured to rotate with regard to the proximal portion the closing direction.

6. The safety device according to claim 3, wherein the pivot link is a living hinge configured to be permanently deformed by the rotation of the distal portion.

7. The safety device according to claim 1, wherein the shield and the ring comprise locking members configured to lock the shield to the ring in the safety position.

8. The safety device according to claim 7, wherein the locking members comprise at least one dead stop defined on the ring and at least one moving stop defined on the proximal portion of the shield and wherein the moving stop is configured to be on a first side of the dead stop in the retracted position of the shield and to move to a second side of the dead stop when the shield is rotated to the safety position.

9. The safety device according to claim 1,wherein the shield has a longitudinal axis and the distal portion has a length along the longitudinal axis of half or less than half a length of the proximal portion along the longitudinal axis.

10. The safety device according to claim 9, wherein the ring is centred on a central axis and the shield is configured so that a longitudinal axis of the distal portion forms an angle of 75 to 105° with the central axis of the ring when the shield is in the safety position and the distal portion is in the tilted position.

11. The safety device according to claim 3, wherein the shield comprises a thinned portion including the pivot link and provided between the proximal portion and the distal portion.

12. The safety device according to claim 11,wherein the shield comprises a main surface and at least a lateral surface, the thinned portion being provided at least on the lateral surface and being configured to fold when the distal portion rotates.

13. A safety needle hub adapted to be fixed on the tip of a medical device, the safety needle hub comprising a needle and a safety device according to claim 1.

14. A medical device comprising a needle and a safety device according to claim 1.

15. The safety needle hub according to claim 13, wherein the needle has a tip and the distal portion of the shield is adapted to cover at least the tip of the needle when the shield is in the safety position.

16. The safety needle hub or the medical device according to claim 15, wherein the distal portion comprises an inside pad adapted to contact the tip of the needle when the distal portion is in the tilted position.

17. The safety needle hub or the medical device according to claim 13,wherein the distal portion of the shield covers at most a distal half portion of the needle when the shield is in the first safety position.
